(19) United States
(12) Patent Application Publication
(10) Pub. No.: US 2004/0170605 A1
(43) Pub. Date: Sep. 2, 2004

(54) Use of the slug gene as a genetic marker in functions mediated by SCF (stem cell factor) and applications

(76) Inventors: Isidro Sanchez Garcia, Salamanca (ES); Jesus Perez Losada, Salamanca (ES)

Correspondence Address: WILLIAM COLLARD, COLLARD & ROE, P.C., 1077 NORTHERN BOULEVARD, ROSLYN, NY, 11576, US

(21) Appl. No.: 10/375,715

(22) Filed: Feb. 27, 2003

(51) Int. Cl. A61K 48/00

(52) U.S. Cl. 424/93.2; 514/44

(57) ABSTRACT

The Slug gene mediates the functions of SCF linking and its c-kit receptor which means that the Slug gene, the Slug gene's CDNA, Slug protein and/or drugs or substances that activate the expression of the Slug gene can be used as therapeutic agents in the mobilization of hematopoyetic stem cells for transplants or gene therapy, in the ex vivo expansion of hematopoyetic stem cells and/or in the treatment of masculine sterility problems.

### FIELD OF THE INVENTION

The invention relates to the use of the Slug gene as a genetic marker for functions mediated by SCF (stem cell factor) and the use as a therapeutic agent of the Slug gene, the said gene's DNA, the Slug protein and drugs or substances that activate the expression of the Slug gene in the mobilization of hematopoyetic stem cells for transplants or gene therapy in the ex vivo expansion of hematopoyetic stem cells and/or the treatment of masculine sterility problems.

### BACKGROUND OF THE INVENTION

Hematopoiesis is a process that produces the replacement of both hematopoyetic progenitor cells and mature blood cells from a reserve of pluripotent stem cells. The daily production of blood cells in a normal adult is precisely regulated, involving a complex interaction between stimulating and inhibiting cytocins, soluble and joined to membranes, and their corresponding receptors. The molecular cloning of these factors of hematopoyetic growth and their receptors has served as an effective instrument for tracing the routes that lead from a single hematopoyetic stem cell to diverse terminally differentiated cells in the hematopoyetic system.

Although numerous cytocins have effects on progenitor and stem cells, in vivo or in vitro, a cytocin discovered at the beginning of the nineties, identified as c-kit, seems to have unique and non-redundant activities on primitive/progenitor cells (Witte, 1990, Cell, 63:5-6). The in vivo functions of c-kit are understood to be either due to the existence of mutant mice in which the genetic code of the receptor (c-kit) and/or its respective linking (SCF) are defective. The mutations in the c-kit receptor and its linking (SCF) are either represented by the existence of numerous mutant alleles with white spots (W) and Steel (S1), respectively.

The mice suffering from mutations in the locus W were originally identified by the presence of white spots on pigmented mice (Silvers, 1979, “Dominant spotting, patch, and rump-white”, in Silvers WK (eds): The coat colors of mice: a model for mammalian gene action and interaction. New York, N.Y., Springer-Verlag, p. 206). A detailed examination of the mice showed that the mutation was pleitropic. The W mice also suffered from defects in the development of germinal cells and hematopoiesis (characterized by macrocitical anemia). It was subsequently shown that the locus W coded a tyrosine kinsase receptor known as c-kit (Nature, 1988, 335:88; Cell, 1988, 55:185).

Before the discovery of locus W, a mutation (S1) was identified in mice with a phenotype that was almost identical to that of W mice (Sarvella and Russell, 1956, J. Hered, 47:123). Since the mutations in two different chromosomes had the same complex phenotype which affected pigmentation, germinal cells and hematopoiesis, the hypothesis was considered that there must be a relationship between the proteins coded in those to loci. In 1990, the protein coded in the locus S1 was identified and denominated as a growth factor in mastocytes, stem cell factor (SCF) and c-kit linking (Cell, 1990, 63:203; Cell, 1990, 63:167; Cell, 1990, 63:175; Cell, 1990, 63;213).

Although the primary function of SCF in early hematopoiesis could be to induce the growth of inactive progenitor/stem cells through synergetic interactions with other early-acting cytocins, there is also ample evidence which shows that SCF, in the absence of other cytocins, stimulates viability selectively prior to the proliferation of murine progenitor cells. Although the SCF/c-kit migratory route and development is well documented, little is known about the molecular mechanisms that provide biological specificity to the SCF/c-kit signaling route in the formation and migration of the different cells from bone marrow.

The biological events controlled by the SCF/c-kit signaling route are similar to those that take place in epithelial-mesenchymal transitions (EMT) in mammal development. In fact, the mesoderm formation process involves the acquisition of migratory properties and the determination of cell destination. These EMT are controlled by a conserved family of proteins of the “zinc finger” type, the Snail family. In fact, the Drosophila Snail gene is vital to the formation of the mesoderm and the destination of cellular migration. The related murine genes (Snail and Slug) have also been present as participants in the formation of the mesoderm and cellular migration.

The use of SCF in the mobilization of hematopoyetic stem cells for transplant or gene therapy and/or in the ex vivo expansion of hematopoyetic stem cells has important side effects and has been limited by its mastocyte-activating properties.

It has now been discovered that the SCF/c-kit signaling route specifically induces the expression of a member of the Snail gene family, the Slug gene, in both natural c-kit cells and in artificially created cells.

### COMPENDIUM OF THE INVENTION

In general, the invention is faced with the problem of finding an alternative method for mobilizing hematopoyetics for gene therapy or transplant and/or in the ex vivo expansion of hematopoyetic stem cells, without the disadvantages mentioned above in relation to the use of SCF.

The solution provided by this invention is based on the identification of the Slug gene as the gene responsible for the functions of the c-kit receptor and its SCF linking. In effect, it has now been discovered that the SCF/c-kit signaling route specifically induces the expression of the Slug gene, a member of the Snail gene family, in both natural c-kit cells and in artificially created cells. As a consequence of the identification of the Slug gene as the gene that mediates SCF linking and c-kit receptor functions, the said Slug gene can replace SCF in its functions and applications, without causing the side effects of SCF associated with mastocyte activation.

Therefore, in view of the applications of SCF, one of the objects of this invention is the use of the Slug gene, the gene's DNA, the Slug protein or substances that activate the expression of the Slug gene, in the preparation of a pharmaceutical compound for the mobilization of hematopoyetic stem cells for transplant or gene therapy.

An additional object of this invention is a method for the ex vivo expansion of hematopoyetic stem cells.

Another additional object of this invention is the use of the Slug gene to prepare a pharmaceutical composition for the treatment of masculine sterility.

The pharmaceutical composition that includes the Slug gene, the Slug gene's DNA, the Slug protein or substances that activate the expression of the Slug gene is an additional object of this invention.

### BRIEF DESCRIPTION OF THE FIGURES

FIG. 1 illustrates the fact that the activation of the c-kit receptor by SCF specifically induces Slug expression. The expression of Slug and Snail was analyzed by RT-PCR in LAMA 84 cells (panels A and B) and in Ba/F3 cells designed to express c-kit (panels C and D) in the absence and presence of SCF. The products of the PCR were transferred to a nylon membrane and analyzed by hybridization with internal oligonucleotide catheters, marked on the ends, specific for each gene (β-actin was used to verify the CDNA load and integrity).

FIGS. 2A and 2B show the defects in the pigmentation and testicles of Slug-deficient mice. FIG. 2A is a photograph of a mutant mouse homocygotic for Slug with the characteristics mark of a white guide on the forehead. FIG. 2B shows the results of the histological analysis of the testicles of wild mice and mice with the Slug mutation. The paired sections of testicles of 6-week wild mice (+/+), heterocygotic mice (Slug +/−), homocygotic mice (Slug −/−), mutant Steel mice (S1/S1), and mutant W mice (W/W) were dyed with hematoxylin and eosin (H&E). There is a generalized reduction in the size of the canaliculus seminifer in Slug −/− mice, a characteristic that is also verified in Slug +/−mice (Slug +/−center as opposed to Slug +/−right). In the interstitial space in Slug −/− mice, there is a reduced quantity of Leydig cells. On the contrary, the interstitial space in the testicles of W/W mice and S1/S1 mice is disproportionately increased and filled with Leydig cells.

FIGS. 3A and 3B show the developmental defects of erythroids in Slug-deficient mice. FIG. 3A shows the results of the histological tests of non-impregnated mice compared to the spleens of 12-day impregnated control mice (Slug +/+), heterocygotic mice (Slug +/−), homocygotic mice (Slug −/−). The tincture with H&E showed, during gestation, an enormous increase in the red pulp of the spleens of Slug +/+mice, in which the white pulp of the spleen, marked with white arrows, was sharply reduced. The increase in red pulp in the spleen was much less evident in Slug +/−and Slug −/− mice. FIG. 3B shows the results of the representative analysis of the c-kit cells present in the bone marrow (BM) and in the spleens of the mice after phenylhydrazine-induced hemolytic anemia. The isolated cells of a wild control mouse (Slug +/+), a mutant heterocygotic mouse (Slug +/−), a mutant homocygotic mouse (Slug −/−), a mutant Steel mouse (S1/S1), and a mutant W mouse (W/W) were dyed with the monoclonal antibody PE-CD117 and analyzed by flow cytometry. The percentage of c-kit cells is indicated.

FIG. 4 illustrates the deficient development of T cells and the apoptosis in the thymus of Slug-deficient mice. A histological analysis was conducted on sections of the thymus of 4-week mutant Steel (S1/S1), mutant W (W/W), wild (control) mice and mutant homocygotic mice (Slug −/− mice). All of the sections were dyed with H&E. The pair sections of Slug −/− mice underwent DAPI OR TUNEL verification. The increased apoptosis in Slug-deficient animals was correlated with atrophy of the thymus. The left side shows a representative analysis of the cells present in the thymus of these mice The isolated cells of a wild control mouse, a mutant heterocygotic mouse (Slug +/−), a mutant homocygotic mouse (Slug −/−), a mutant Steel mouse (S1/S1), and a mutant W mouse (W/W) were dyed with the monoclonal antibody and analyzed by flow cytometry. The percentage of cells is indicated.

FIGS. 5A-5C illustrate the development of B cells, myeloid and mastocytes in Slug-mutant mice. The results of a representative analysis of the B cells and myeloid present in the spleen (FIG. 5A) and in the bone marrow (BM) (FIG. 5B) are shown. The isolated cells of a wild control mouse, a mutant heterocygotic mouse (Slug +/−), a mutant homocygotic mouse (Slug −/−), a mutant Steel mouse (S1/S1), and a mutant W mouse (W/W) were dyed with the monoclonal antibody and analyzed by flow cytometry. The percentage of cells is indicated. FIG. 5C shows the results of a histological analysis of 4-week sections of mutant homocygotic mice (Slug −/−) and mutant W mice (c-kit −/−). All of the sections were dyed with Giemsa. The arrows indicate the presence of mastocytes in −/− Slug-mutant mice but not in W mutant mice.

FIG. 6 shows that the defect in Slug-mutant mice is intrinsic to the stem cell. Panel 6A shows the results of kit immunoprecipitations using isolated mastocytes tested for kit and re-tested for phosphotyrosine. Slug +/−heterocygotic mice, Slug +/+homocygotic mice, wild mice. Panel 6B shows the results of an analysis of the hematopoyetic system in normal receptor mice reconstituted with Slug −/− HSC by FACS. The samples of the bone marrow, spleen and thymus were dyed with monoclonal antibodies and analyzed by flow cytometry. The hematopoyetic composition is similar to that of Slug −/− mice. To the left is a representation of the c-kit cells present in the bone marrow (BM) and in the spleen of normal receptor mice reconstituted with Slug −/− HSC after phenylhydrazine-induced hemolytic anemia.

FIGS. 7A-7C illustrate the signaling route of SCF/c-kit in development. FIG. 7A shows the c-kit cells present in the bone marrow (BM) and the spleen of wild (control) mice, mutant Steel mice (S1/S1), and mutant W mice (W/W) after phenylhydrazine-induced hemolytic anemia. For cell separation and classification, the cells were incubated with c-kit PE cells and the c-kit cells were classified by fluorescent activation (FACS) (FACstar, Becton Dickinson). The classified cells were then analyzed again to determine the level of purity with the cytometer, obtaining a purity level of ≧95%. FIG. 7B shows the results of the Slug expression, analyzed by RT-PCR, of the purified c-kit cells of the bone marrow and spleen of control mice, S1/S1, and W/W. The products of the PCR were transferred to a nylon membrane and analyzed by hybridization with internal oligonucleotide catheters, marked on the ends, specific for each Slug gene (upper panel). β-actin was used to verify the CDNA load and integrity (lower panel). FIG. 7C is an illustrative outline of a model for the function of the SCF/c-kit signaling route in development. The SCF/c-kit signaling route affects the development of three cell populations: melanoblasts, hematopoyetic stem cells and germinal cells. The data indicate that Slug mediates the c-kit receptor and linking functions in melanoblasts and in hematopoyetic cells. In germinal cells, the SCF/c-kit function is mediated by Slug and by P13-kinase.

### DETAILED DESCRIPTION OF THE INVENTION

In general, the invention refers to the use of the Slug gene as a genetic marker of functions mediated by SCF and as a therapeutic agent of the Slug gene, the complementary DNA (CDNA), the RNA that codes for the product of the transcription or expression of the said Slug gene (hereinafter, CDNA of the Slug gene), the product of the expression and translation of the Slug gene (hereinafter, Slug protein) and drugs or substances that activate Slug gene expression in mobilizing hematopoyetic stem cells for gene transplant or therapy in the ex vivo expression of hematopoyetic stem cells and/or for the treatment of masculine sterility.

The Slug gene is a gene present in vertebrates that codes for a transcription factor of the “zinc fingers” type (SLUG), implicated in epithelial-mesenchymal transitions (Nieto et al., Science 264: 835-849 (1994)). Surprisingly, it has now been discovered that the Slug gene is responsible for the functions of the c-kit receptor and its SCF linking. Consequently, said Slug gene and the CDNA of the said Slug gene, Slug protein and the drugs or substances that activate the expression of the Slug gene may be used in the same applications as SCF, without the side effects of SCF associated with the mastocyte activation.

In order to ascertain the molecular mechanisms that provide biological specificity to the SFC/c-kit signaling route in the formation and migration of different cells from the bone marrow, the relationship between the SFC/c-kit signaling route and the Snail protein family has been investigated, with the surprising discovery that the SFC/c-kit signaling route specifically induces the expression of the Slug gen in both natural c-kit cells and artificially created cells. The analysis of a directed null mutation that eliminated all of the Slug's coding sequences revealed that mutant Slug mice, as well as c-kit and SFC defective mice, have a complex phenotype that includes pigmentation, gonadal and hematopoyetic defects.

As used in this description, the term “mutant Slug mice” refers to mice with a different phenotype than the original (wild type, Slug +/+) due to a mutation in the Slug gene, and includes both heterocygotic mutant mice (Slug +/−) and homocygotic mutant mice (Slug −/−).

Long term transplant experiments demonstrated that the effect in mutant Slug mice, in which the c-kit cells of Slug −/− mice have a functional SCF/c-kit signaling route, presented migratory defects similar to those of the c-kit cells in S1/S1 and W/W mice, which is intrinsic to the cell. Since mutations in SCF, in the c-kit receptor and in the Slug gene have a similar pleiotropic phenotype that affects pigmentation, germinal cells and hematopoiesis, the hypothesis was drawn that there must be some relationship between hem In fact, two different pieces of data demonstrated the relationship. First of all, the primary c-kit cells purified from the control mice express Slug. Secondly, the Slug gene is not expressed in the primary c-kit cells derived from W/W and S1/S1 mice. These two results combined identify the Slug gene as the molecular objective that provides biological specificity to the SCF/c-kit signaling route.

The invention provides a pharmaceutical composition that comprises the Slug gene, the Slug gene's CDNA, the Slug protein and/or one or more drugs or substances that activate the expression of the Slug gene, along with one or more pharmaceutically acceptable excipients. The Slug gene, the Slug gene's CDNA and the Slug protein can be obtained using conventional genetic engineering techniques (see Example 1). The drugs or substances that activate the expression of the Slug gene can be obtained using conventional techniques which include, for example, the preparation of DNA constructions that include the Slug gene or the Slug gene's CDNA and a delator gene which, when it comes into contact with the drug or substance being tested, makes it possible to determine whether the said drug or substance activates the expression of the Slug gene. By way of example, the delator gene may be the gene that codes for a protein for which there are specific antibodies, or a protein with enzymatic activity such as GFP. The activity is detected by adding the pertinent substrate or developing system

The excipients that may be used in the pharmaceutical composition of the invention will depend, among other things, on the manner in which the pharmaceutical composition is administered. A review of the different ways of administering active ingredients, of the excipients to be used and the manufacturing procedures may be found in the Tratado de Farmacia Galenica, C. Fauli i Trillo, Luzân 5, S.A. de Ediciones, 1993.

When the pharmaceutical composition of the invention contains the Slug gene or the Slug gene's CDNA, the pharmaceutical composition will include certain vectors or systems that aid the transfer process from an exogenous gene to a cell, facilitating the delivery and intracellular bioavailability of the gene so that it can function properly. For example, these vectors may be viral vectors such as those based on retroviruses or adenoviruses, or non-viral such as DNA-liposome, DNA-polymer, DNA-polymer-liposome compounds, etc. [see, “Nonviral Vectors for Gene Therapy”, edited by Huang, Hung and Wagner, Academic Press (1999)].

It is known that both bone marrow transplants and gene therapy strategies use hematopoyetic stem cells as target cells. Since the number of hematopoyetic stem cells in peripheral blood is limited, it is necessary to mobilize hematopoyetic stem cells from the bone marrow to peripheral blood by different means, such as bone marrow transplant, gene therapy, ex vivo manipulation of hematopoyetic stem cells, etc. Surprisingly, it has now been found that the Slug gene, the CDNA of the Slug gene, the Slug protein and/or drugs or substances that activate the expression of the Slug gene may be used to mobilize hematopoyetic stem cells.

Therefore, another aspect of the invention refers to the use of the Slug gene, the Slug gene's CDNA, the Slug protein and/or drugs or substances that activate the expression of the Slug gene to prepare pharmaceutical compositions to mobilize hematopoyetic stem cells for transplant or gene therapy. The hematopoyetic stem cells are mobilized in the transplant recipient or in the patient undergoing gene therapy.

The Slug gene, the CDNA of the Slug gene, the Slug protein and/or drugs or substances that activate the expression of the Slug gene may enable the ex vivo survival of hematopoyetic stem cells, which favors their maintenance and manipulation.

On the other hand, it is known that a decrease in Leydig cells causes fertility problems, particularly masculine sterility. Surprisingly, it has now been observed that the Slug gene favors the migration and/or survival of Leydig cells. Therefore, the administration to male patients suffering from sterility in need of treatment of a pharmaceutical composition provided by this invention that contains the Slug gene, the Slug gene's CDNA, the Slug protein and/or one or more drugs or substances that activate the expression of the Slug gene, along with one or more pharmaceutically acceptable excipients, may solve certain masculine sterility problems.

Therefore, another aspect of the invention refers to the use of the Slug gene, the Slug gene's CDNA, the Slug protein and/or drugs or substances that activate the expression of the Slug gene to prepare pharmaceutical compositions for the treatment of masculine sterility, particularly for the treatment of masculine sterility brought on by a decrease in Leydig cells.

The invention is illustrated below by means of a trial that illustrates how the expression of the Slug gene is induced by the activation of the c-kit receptor by SCF.

### EXAMPLE 1

Expression of the Slug gene induced by activation of the c-kit receptor by SCF.

### Materials and Methods

### Cell Culture

The cell lines used include LAMA-84 and Ba/F3 cells. The cells were kept in a modified Dulbecco Eagle (DMEM) medium supplemented with 10% fetal calf serum (FCS). When necessary, a conditioned WEHI-3B medium was added as a sources of interleukin 3 (IL-3). The Ba/F3 cells that express the c-kit wild type were obtained as described below. The PECE-kit plasmid that contains the complete coding sequence of the mouse's c-kit CDNA (donated by Dr. D. Martin-Zanca) was used to transfect the pro-B IL-3-dependent cell line. The co-transfection with a neomycin-resistant plasmid (MCI-neo) and the primary selection with the analogue of neomycin G418 generated a stable Ba/F3 cellular line that expresses c-kit (Ba/F3+c-kit). The populations of Ba/F3 cells were died with the c-kit-specific CD117 monoclonal antibody.

### Mice

The animals were caged under non-sterile conditions in a conventional animal facility. The heterocygotic and homocygotic mice for the Slug^{Δ1} mutation generated by the suppression of the genome sequences of the entire coding region of the Slugh protein (mutant Slug^{Δ1} mice) have been described previously (Jiang et al, 1998, Developmental Biology 198:277-285). The W/W and S1/S1 mice and the pairs of breeding pairs were obtained from Jackson Laboratory (Bar Harbor, Me.). The experimental mice were injected intraperitoneally with phenylhydrazine (PHZ; 60 mg per kilogram of body weight; Sima Chem) for two consecutive days (Broudy et al, 1996, Blood 88:75-81). On each one of the three days following the second PHZ injection, 5 mice died of cervical dislocation and their bones and spleens were removed (under sterile conditions) for further analysis. All procedures were approved by the institutional animal care committee.

### Phenotypical Cell Analysis

The cellular morphology was analyzed according to standard criteria. The unicellular suspensions were prepared from individual tissues, including bone marrow, spleen, thymus and peripheral blood using standard procedures (Garcia-Hernandez et al, 1997, PNAS). For most of the tinctures approximately 1×10⁶ cells were used. The phenotypes of the cells were immunized with the following antibodies: conjugated (PE)TER119 (Ly-76, a monoclonal antibody that recognizes an antigen expressed in erythroid cells from erythroblasts to erythrocytes); PE-CD4, PE-Gr-1, PE-CD117, PE-CD19, PE-B220, conjugated fluorescein (FITC)-CD8, FITC-IgM, FITC-MacI (all from Pharmingen). Cells, suspended in a (PSB) phosphate saline solution, without Ca** or Mg** with 1% (v/v) fetal bovine serum, were marked with each antibody (approximately 1 μg/10⁶ cells) for 30 minutes on ice. Cellular fluorescence was analyzed with the FACScan (Becton Dickinson) cytometric flow. The incubated cells with properly marked isotopic controls (Pharmingen) were used to avoid the output of non-specified fluorescence signals. Before the analysis, the mature red cells were reduced by hypotonic breakage (0.38% ammonium chloride for 15 minutes on ice). The base controls were handled in the same way, with the exception that the primary antibodies were omitted. Initially, the cells were analyzed by size and by dispersion to identify the live cells. In some experiments, cellular viability was evaluated by exclusion with propidium iodide (5 μg/ml, Sigma) in flow cytometry).

### Cellular Purification

The mononuclear spleen suspensions were prepared by cutting the spleens into small fragments in 5 ml of PBS solution without CA** or MG**, containing 10% FBS (v/v) and passing the cellular suspension through progressively smaller needles. The bone marrow cells were removed from the femurs with a syringe containing 2 ml of PBS with 10% FBS. The low density mononuclear cells of the bone marrow and spleen were isolated by subjecting them to centrifuging over Ficoll-Paque (P=1,077 g/ml) at 800 grams for 20 minutes at room temperature. For cell classification, the cells were incubated with c-kit-PE and the c-kit cells were classified using a cellular classifier activated by fluorescence (FACS) (FACstar, Becton Dickinson). The classified cells were analyzed again with cytometry to determine their purity.

### Retrotranscription Polymerase Chain Reaction (RT-PCR)

To analyze the expression of Slug and Snail in the cell lines and in the purified c-kit cells, an RT was performed in accordance with the manufacturer's protocol in a reaction of 20 μl that contained 50 ng of random hexamers, 3 μg of total RNA and 200 units of Superscript II RNAse H reverse transcriptase (GIBCO/BRL). The parameters of the thermal cycles for the PCR and the sequences of the specific primers were as follows: SLUG, 30 cycles at 94° C. for 1 minute, 56° C. for 1 minute and 72° C. for 2 minutes, primer in correct sense, 5′GCCTCCAAAAAGCCAAACTA3′ and antisense primer, 5′CACAGTGATGGGGCTGTATG-3′ mSnail, 30 cycles at 95° C. for 2 minutes, 60° C. for 2 minutes and 72° C. for 2 minutes primer in correct sense, 5′CAGCTGGCCAGGCTCTCGGT-3′ and antisense primer, 5′GCGAGGGCCTCCGGAGCA-3′. The amplification of the mRNA of β-actin served as a control to evaluate the quality of each sample of RNA. The sequences of the internal probes were the following: mSlug, 5′GACACACATACAGTGATTATTTCC-3′ and mSnail, 5′TGCAACCGTGTTTGCTGACCGCTCCAAC-3′.

### Bone Marrow Transplant (BMT) and Sample-Taking

The female receptor mice C57BL/61 (8-12 weeks old) were irradiated with two divided doses of 600 cGy two hours apart. This dose is sufficient to completely eliminate the hematopoiesis endogens. BM cells were injected into the tail veins of the radiated mice a 2-4×10⁶ cells per mouse for long term reconstitution. All receptors were kept in isolated cages with acidified sterilized food and water. The animals were slaughtered and the hematopoyetic tissue collected for FACS analysis.

### Hematopoyetic Colony Tests

The bone marrow cells (0.25-1.0×10⁶ cells/plate) and the spleen cells (10⁴-10⁵ cells/plate) isolated from the normal mice and Slug-mutants were placed on semi-solid culture plates free of FBS (Stem Cell Technology). The growth of the colony was stimulated with the following combinations of recombinant growth factors: mouse stem cell factor (100 ng/ml; SIGMA), IL-3 for mice (10 ng/ml, SIGMA), and human erythropoyetin (hEPO) (2 U/ml, ROCHE) for the growth of the forming unit of the enthroid ramification (BFU-E). The growth of the colonies derived from the forming unit of enthroid colonies (CFU-E) was stimulated with EPO (2 U/ml). The cultures were incubated at 37° C. in a humidified incubator containing 5% CO₂ in the air and the results were observed after 3 days (for colonies derived from CFU-E) or 7 days (for colonies derived from BFU-E) following the initiation of the culture. The frequency of BFU-E and CFU-E was determined in the cultures in triplicate.

### Isolation of Primary Mastocytes Derived from Bone Marrow, Immunoprecipitation and Western-Blotting

The bone marrow cells were collected by irrigating the femur bone cavity and he mastocytes were collected by selective growth in a medium that contained IL-3 for 6 weeks (Opti-Mem I, GIBOCOBRL 10% fetal bovine serum, 0.5 ng/ml of IL-3 recombinant murine, R&D Systems Inc.). The medium was replaced every day and the cells were transferred to new plates to eliminate the stuck cells, including macrophagie and megacariocytes. The immunoprecipitation and western blotting tests were conducted using extracts of 1×10⁷ mastocytes per band. The cells were deprived of food for 12 hours in an Opti-Mem I medium without IL-3 containing only 0.5% serum before being stimulated with 100 ng/ml of SCF murine (R&D Systems, Inc.) for 10 minutes at 37° C., as indicated. Kit was detected using a goat anti-serum purified by affinity opposite the C-terminal end of the murine kit receptor, M-14 (Santa Cruz). The monoclonal antibody 4G10 (UBI) was used to detect phosphotyrosine.

### Histological Analysis

The tissue samples were set overnight in 10% formalin and then processed. They were soaked in paraffin and 6 μm sections were dyed with hematoxylin and eosin. They were examined histologically and photographed. All of the sections were taken from homogeneous and viable portions of the cut tissue. The mastocytes were dyed with Giemsa. The number of mastocytes per square millimeter was determined. TUNEL Test

Terminal deoxynucleotidyltransferase-mediated dUTP-biotin Nick End Labeling was conducted using the in situ dead cell detection kit (Boehringer Manhein), essentially following the manufacturer's instructions with some minor modifications depending on the type of preparation. Briefly, the sections were subsequently set for 15 minutes in 4% paraformaldehyde, rinsed twice with PBS and incubated in a 2:1 mixture of ethanol and acetic acid for 5 minutes at −20° C. After 2 PBS rinses, the sections were subjected to digestion in K proteinase (10 μg/ml in 10 mM Tris HCl, pH 8.0 and EDTA 1 mM), rinsed twice with PBS and countercolored with methyl green.

### II. RESULTS

### Induction of Slug Expression Through the Activation of the c-kit Receptor by SCF

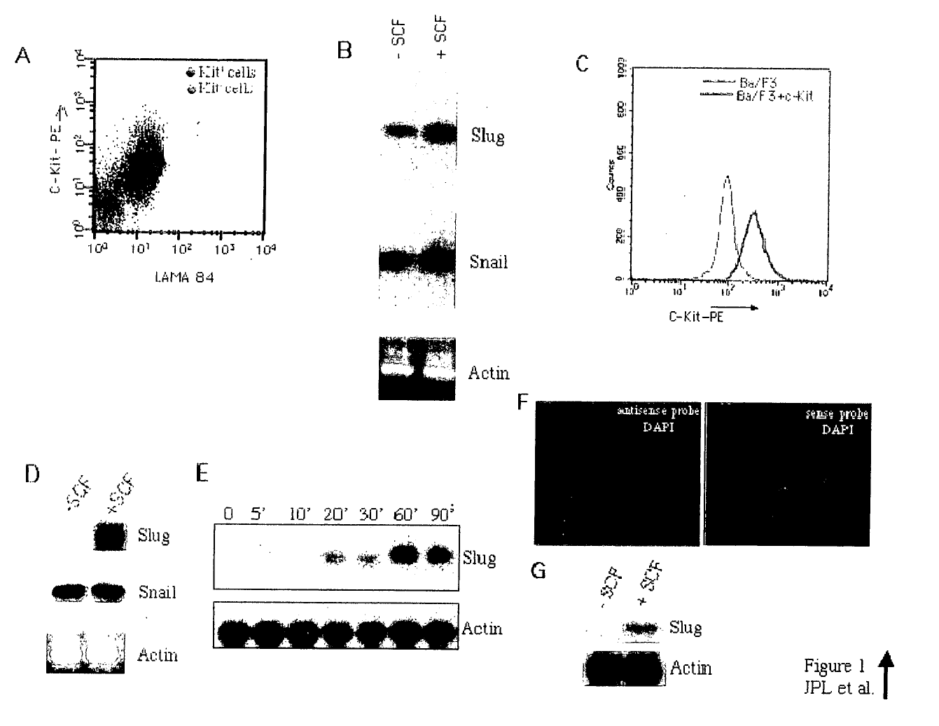

The ability of the c-kit receptor to stimulate the expression of members of the Snail family was tested primarily on c-kit* cells expressed naturally, using the LAMA 84 cell line (FIG. 1A). As shown in FIG. 1B, the expression of Slug increased rapidly in the LAMA 84 cells treated with SCF. However, the level of Snail expression was not modified in the presence of SCF. To a certain extent, these preliminary data indicate the ability of LAMA 84 cells treated with SCF to specifically activate the expression of the Slug gene. The Ba/F3 cells that were missing the c-kit endogen (Palacios and Steinmetz, 1985, Cell 41:727) were manipulated to express a wild type c-kit receptor and complete length (Ba/F3+c-kit) (FIG. 1C). The c-kit-transfected cells specifically expressed Slug with the SCF stimulation (FIG. 1D). However, the Snail gene was expressed at similar levels in Ba/F3+c-kit cells not stimulated by SCF and in Ba/F3+c-kit cells stimulated by SFC. These experiments demonstrate that the activation of c-kit specifically induces the expression of Slug, thus indicating a clear relationship between the activation of c-kit/SCF and the expression of Slug. Due to the fact that the mutations in two different genes, the c-kit receptor and its linking (SCF) have the same complex phenotype that affects pigmentation, germinal cells ad hematopoiesis, the mice that did not have the Slug gene were analyzed thoroughly to determine whether the functions of the c-kit/SCF route in vivo were mediated by Slug.

Pigmentation, Gonadal and Hematopoyetic Defects in Slug-Mutant Mice.

The most obvious phenotype of in vivo S1 and W mutants is the presence of severe dwarfism, which is observed shortly after birth. This characteristic is also observed in the mice that carry a null mutation of the Slug gene (homocygotic Slugh^{Δ1} mutant mice), which looked considerably smaller than the rest of the litter (Jiang et al, 1998, Developmental Biology 198:277-285).

As in c-kit and SCF-defective mice, the delay in the growth of homocygotic Slugh^{Δ1} mutant mice occurred during the first three weeks of life. The Slug gene was therefore then studied to determine whether the gene, as the c-kit and linking (SCF) receptor are also important in dermal, gonadal and hematopoyetic development.

### 1. Pigmentation Deficiencies

The melanoblasts originate in the pluripotent neural crest and emigrate along certain characteristic paths. They depend on numerous signaling systems for both their survival and migration (Ling et al, 2000, Development 127:5739-5389). The mutant heterocygotic mice (W/+ or S1/+) have a characteristic white spot on the forehead and additional areas of depigmentation in the ventral area, tail and paws. The mutant homocygotic mice (W/W or S1/S1) are much more affected, completely lacking any pigmentation in the skin or hair, whose melanocytes derive from the neural crest.

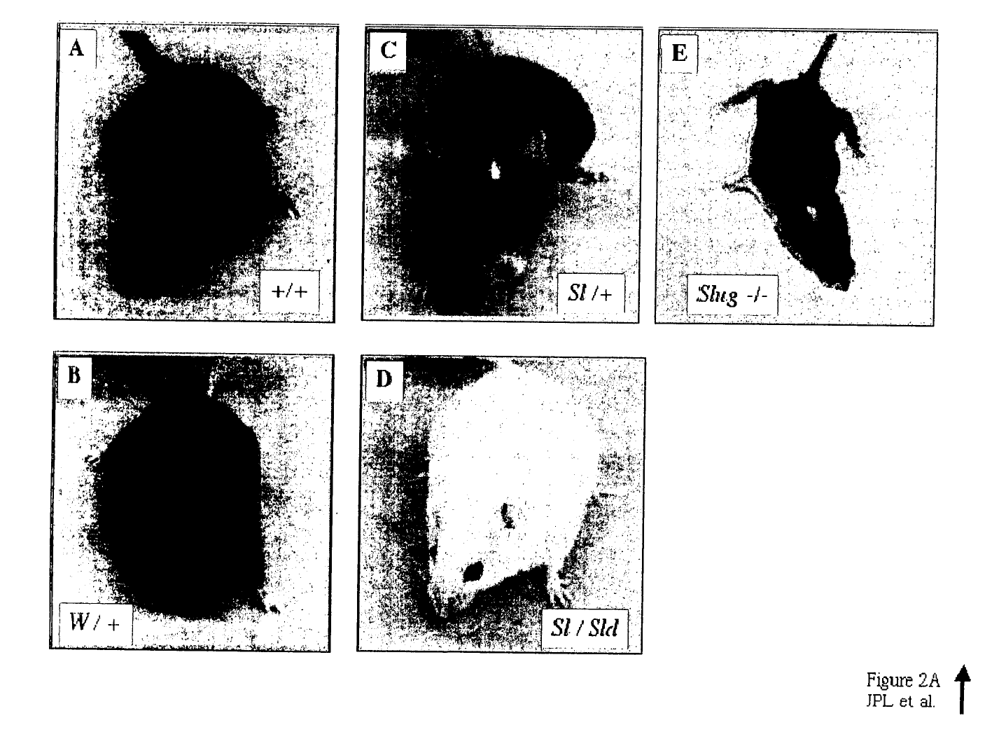

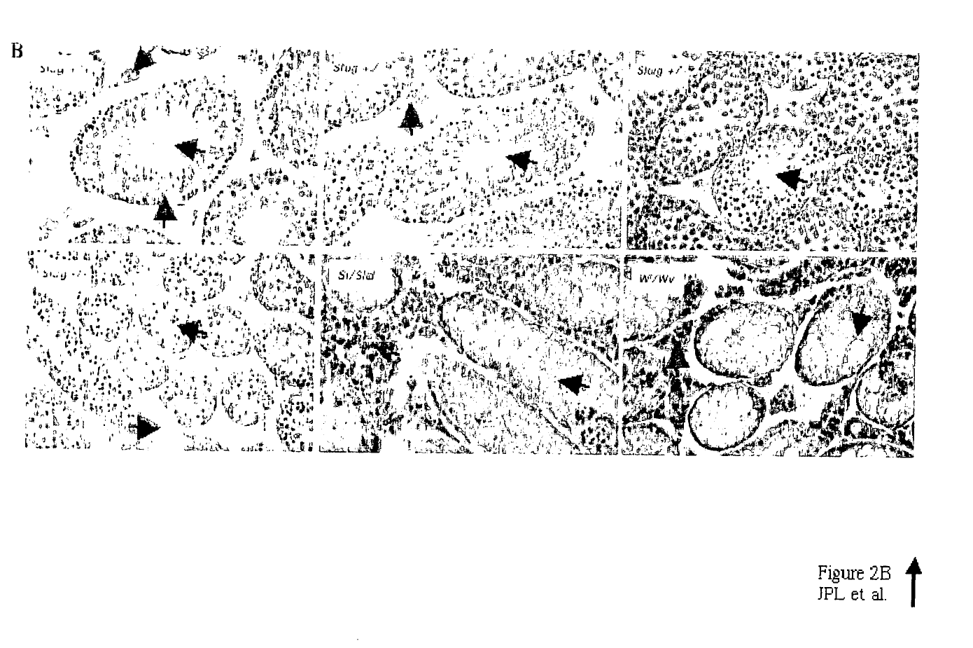

The heterocygotic mice for Slug did not exhibit pigmentation alterations. However, the mutant homocygotic mice for Slug had diluted coats with additional areas of depigmentation on tails and paws and the characteristic white spot on the forehead (FIG. 2A). These dermal defects in Slug −/− mice consisted of several degrees of depigmentation.

However, the retina and internal layer of the iris, whose melanocytes come from the optical bone and are independent from the SCF/c-kit signaling path, are systematically pigmented in Slug −/− mice. These dermal defects observed in Slug −/− mice are similar to the dermal phenotype observed in W/+ and S1/+ and suggest a function of the Slug gene in the development of the melanocytes from the neural crest.

### 2. Gonadal Deficiencies

The Slug-deficient females were fertile and the ovaries looked normal. Most of the Slug −/− males were also fertile. While they appeared to copulate normally, as indicated by the formation of vaginal tampon, more than 15% were unable to induce pregnancy in their partners. The Slug −/− mice that were capable of procreating produced small litters (3-6 mice as opposed to a normal litter of 10-12 mice). The size and weight of the testicles of −/− mutants approximately 40% smaller when compared to the members of litter of wild mice. The histological sections of the testicles of 6-week Slug-deficient mice revealed that the testicular atrophy came from an overall reduction in the size of the seminal tubes, a characteristic that can also be observed in some heterocygotic mice for Slug (FIG. 2B). However, sperm was visible in the lumen in keeping with the fact that fertility was not seriously compromised in these animals. The histological analysis also revealed a reduced number of Leydig cells in the interstitial space in Slug-deficient mice (FIG. 2B). On the contrary, the interstitial space in the testicles of W/W and S1/S1 mice is disproportionately augmented and full of Leydig cells. The Slug gene therefore has a function in the development of the germinal cells in males, but the loss is insufficient to completely compromise the production of sperm cells.

### 3. Hematopoyetic Deficiencies

The mice with null SCF and c-kit mutation have severe hematopoyetic deficiencies. SCF acts on the hematopoyetic progenitor cells, where an increase in the survival more than recruitment was observed within the cellular cycle. Consequently, the function of Slug in normal hematopoiesis was analyzed.

### 3.1. Macrocitical Anemia in −/− Slug Mice

Anemia is the most notable hematopoyetic phenotypical anomaly observed in S1 and WW mutants in vivo and is responsible for stunting growth during the first weeks of life, a characteristic shared with Slug-deficient mice. The blood parameters in mutant Slug −/− mice were then examined. The hematopoyetic parameters examined, in particular hemoglobin (HGB), mean corporal volume (MCV) and mean concentration of corpuscular hemoglobin (MCHC), define a macrocitical anemia (Table 1), an aspect of S1 and W mice and of the human piebald phenotype due to mutations caused by the losses of function that occur naturally in the c-kit receptor and its SCF linking, respectively.

TABLE I
Hematological parameters of the peripheral blood of
Slug +/+, +/− and −/− mice

| | Genotype | | |
|---|---|---|---|
| | −/− | +/− | +/+ |
| RBC (×10⁶/μl) | 8.3 ± 1.1 | 9.15 ± 1.0 | 10.2 ± 0.6 |
| HGB (g/dl) | 11.9 ± 1.6 | 14.8 ± 1.4 | 15.2 ± 0.9 |
| HCT (%) | 37 ± 3.7 | 48.3 ± 4.1 | 48.3 ± 3.4 |
| MCV (fl) | 55.5 ± 4.1 | 48.2 ± 3.1 | 49.3 ± 3.4 |
| MCH (pg) | 19.4 ± 1.4 | 18.3 ± 1.2 | 18.5 ± 1.1 |
| MCHC (g/dl) 34.9 | 38.9 ± 3.5 | 36.7 ± 3.3 | 35.6 ± 3.2 |
| RDW (%) | 15.2 ± 1.3 | 12.2 ± 0.9 | 12.4 ± 1.1 |
| Plaq (×10³/μl) | 422 ± 24.5 | 437 ± 30.8 | 445 ± 34.4 |
| MPV (fl) | 5.2 ± 0.3 | 5.5 ± 0.3 | 5.4 ± 0.4 |
| WBC (×10³/μl) | 8.4 ± 1.0 | 9.07 ± 1.2 | 11.5 ± 1.4 |
| Neu (% N) | 64.6 ± 3.9 | 65.9 ± 4.3 | 66.2 ± 4.7 |
| Lym (% L) | 35.4 ± 2.2 | 33.6 ± 2.9 | 33.8 ± 2.8 |
| Mono (% M) | ND | ND | ND |
| Eos (% E) | ND | 0.5 | ND |
| Baso % B) | ND | ND | ND |

Mean value ± SEM (standard sample error for n=10); RBC, enthrocytes; HGB, hemoglobin; HCT, hematocrit; MCV, mean erythrocyte volume; MCH, mean corpuscular hemoglobin; MCHC, mean concentration of corpuscular hemoglobin, RDW, erythrocyte distribution width; Plaq, platelets; MPV, mean platelet volume; WBC, leukocytes; Neu, neutrophils; Lym, lymphocytes; Mono, monocytes; Eos, eosinophils; baso, basophils; N.D., not detected.

The expansion capacity of enthropoesis in Slug-mutant mice under hematopoyetic stress was then studied. The vast expansion of the enthropoesis that takes place in the spleen of the mice in response to hemolytic anemia or other situations of hematopoyetic stress (during gestation) is due to the migration of BFU-E from the marrow to the spleen. Therefore, the effects of the erythropoiesis on the red pulp of the spleens of the Slug-mutant mice during gestation was first examined. Gestation in mice is characterized by transitory splenomegaly in the middle of gestation due to a sharp increase in the number of erythroblasts. This gestation-associated anemia is he main reason for the change in the size and cellular content of the maternal spleen (Table II). On the contrary, the spleens of Slug-mutant mice at 12 days of gestation are small than those of control mice (Table II).

TABLE II
Weight (in grams) of the spleens of pregnant +/+, +/−
and −/− Slug mice

| | Genotype | | |
|---|---|---|---|
| | −/− | +/− | +/+ |
| Not pregnant | 0.0726 ± 0.0033 | 0.0697 ± 0.0048 | 0.0706 ± 0.0029 |
| Pregnant | 0.1379 ± 0.0075 | 0.0864 ± 0.0039 | 0.0771 ± 0.0029 |

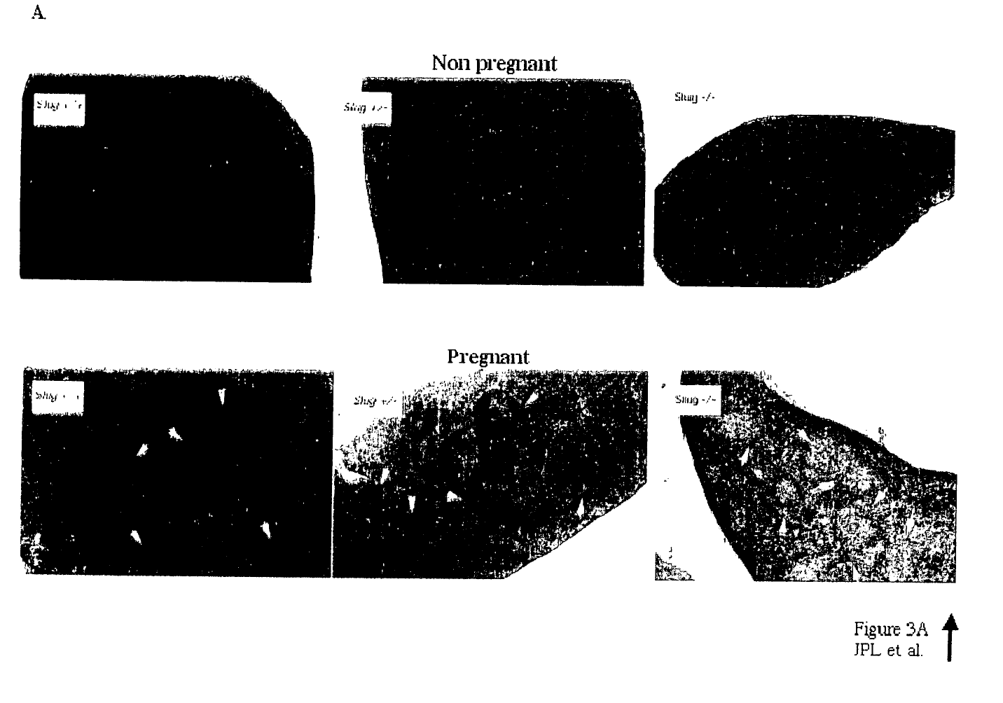

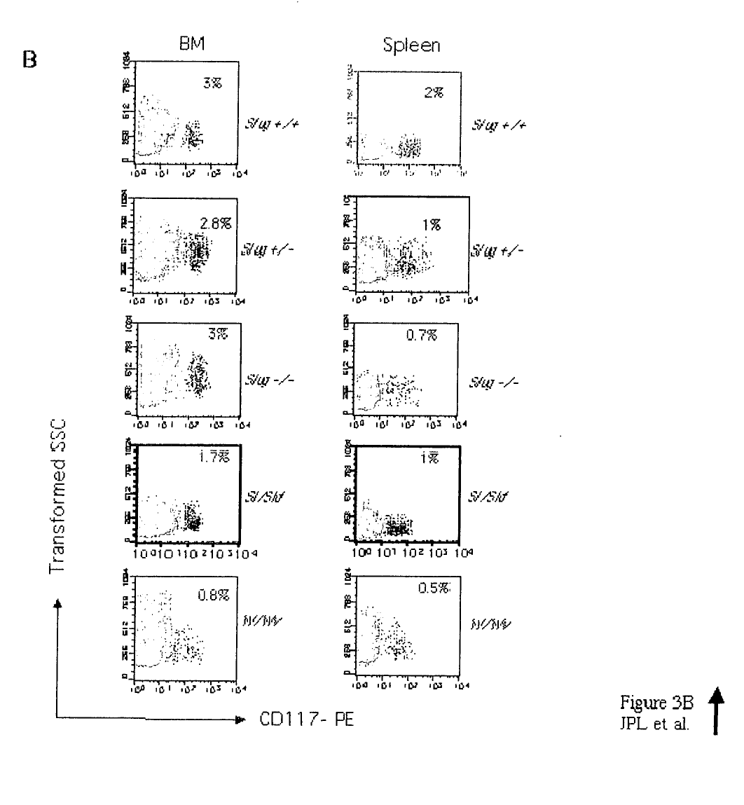

The histological examination of the spleens showed that the increase in the red pulp of the spleen was much less evident in Slug +/−mice than in −/− mice (FIG. 3A). The flow cytometry results of the analysis of the expression of the enthroidal marker (TER-229) in the bone marrow and spleen cells of normal mice and pregnant Slug-mutant mice is shown on Table III.

TABLE III
Frequency (percentage) of TER-110* cells in the bone
marrow and spleen cells of Slug +/+, +/− and −/− mice and
in mice that have recovered from gestation-induced anemia

| | Bone marrow | Spleen |
|---|---|---|
| +/+ | 7.7 ± 1.5 | 1.2 ± 0.9 |
| +/+ during gestation | 19.1 ± 2.1 | 18.3 ± 2.9 |
| +/− | 7.5 ± 1.7 | 1.1 ± 0.8 |
| +/− during gestation | 12 ± 2.2 | 6.0 ± 1.5 |
| −/− | 7.9 ± 1.1 | 1.3 ± 0.7 |
| −/− during gestation | 10 ± 2.1 | 4.9 ± 1.6 |

The frequency of TER-119* cells in increased in both the bone marrow and the spleen during the recovery from gestation-induced anemia in control mice. On the contrary, the increase in TER-119* cells was affected in both the bone marrow and spleens of Slug-mutant mice. These results show the poor recovery from gestation-induced anemia in Slug-mutant mice, indicating a defect in the generation and/or migration of erythroid progenitor cells in Slut-mutant mice. Therefore, the number of BFU-E was then quantified, which is the most primitive erythroid progenitor cell, and of CFU-E, which are the most differentiated progenitor cells, by testing the formation of hematopoyetic colonies in the bone marrow and the spleen of control mice and Slug-mutant mice under physiological conditions (without erythroidal stress). The number of BFU-E and CFU-E cells in heterocygotic mice for Slug was similar to that of the control mice. However, the number of BFU-E in the bone marrow and the number of CFU-E in the spleen had not been reduced in homocygotic mice for Slug in comparison to control mice. These results indicate a basal erythroidal defect at the BFU-E level in Slug −/− mice. However, the basal erythroidal development appears to be normal in Slug −/− mice, although the hematopoyetic stress (gestation) showed little recovery from the anemia.

The number of BFU-E and CFU-E cells was then quantified in the Slug-mutant mice in which hemolytic anemia had previously been induced with phenylhydrazine (PHZ). The injection of PHZ causes a serious destruction of red corpuscles followed by an expansion of the erythropoiesis. Consequently, mice of the same age were injected with PHZ and its effects were systematically observed around day 3 in the mice that had been injected with PHZ, causing a rapid reduction in hematocrit and an increase in the number of reticulocytes (data not demonstrated). In the Slug +/−mice with hemolytic anemia induced by PHZ, the number of CFU-E cells was reduced in BM compared to control mice and the increase in the number of BFU-E and CFU-E cells in the spleen was affected (Table IV). The induction of hemolytic anemia with PHZ in Slug −/− mice resulted in an increase in bone marrow erythropoiesis, but the expected increase in the erythropoiesis of the spleen was not completely blocked (Table IV). These results demonstrate that one of the results of the response to the erythropoyetic demand is the expansion of erythropoiesis, primarily at the BFU-E level, in Slug-mutant mice. A similar phenotype is observed in W/W mice. While the bone marrow erythropoiesis increases around day 3 in the W/W mice to whom PHZ has been administered, the expected increase in spleen erythropoiesis did not occur until day 3. The flow cytometry results of the analysis of the expression of the c-kit marker (CD117) in the bone marrow and spleen of control mice, Slug-mutant mice, S1 mutants and W mutants after the induction of hemolytic anemia with PHZ showed that the increase in c-kit cells in the spleens of Slug-mutant, S1/S1 and W/W mice was blocked in comparison to control mice (FIG. 3B). These results show that the Slug-deficient c-kit cells behave like S1− and W-c-kit cells and the defect in erythroidal development is similar in Slug-mutant, W/W ad S1/S1 mice.

TABLE IV
Expansion in the number of BFU-E and CFU-E cells in the
bone marrow (BM) and spleens of mice treated with
phenylhydrazine (PHZ).

| | No. of CFU-E (×10⁴) | | No. of BFU-E (×10³) | |
|---|---|---|---|---|
| (+/+) | 4.3 ± 0.6 | 6.6 ± 1.5 | 4.2 ± 0.5 | 1.3 ± 0.4 |
| PHZ (+/+) | 16.2 ± 1.1 | 256 ± 29 | 4.4 ± 0.4 | 25 ± 2.4 |
| (+/−) | 3.9 ± 1.3 | 4.1 ± 0.8 | 4.2 ± 0.3 | 1.6 ± 0.2 |
| PHZ (+/−) | 4.1 ± 0.9 | 23 ± 3 | 4.9 ± 0.6 | 11 ± 0.8 |
| (−/−) | 4.0 ± 1.3 | 2.4 ± 0.5 | 1.6 ± 0.1 | 1.5 ± 0.2 |
| PHZ (−/−) | 4.1 ± 1.4 | 2.0 ± 0.4 | 4.6 ± 0.3 | 1.7 ± 0.4 |

The numbers of BFU-E and CFU-E in the bone marrow and spleens of the mice treated with PHZ and slaughtered on day 3 were quantified using hematopoyetic colony formation tests. The values shown represent the average ± SEM of 5 mice from each group.

### 3.2 T Cells in Slug-Mutant Mice

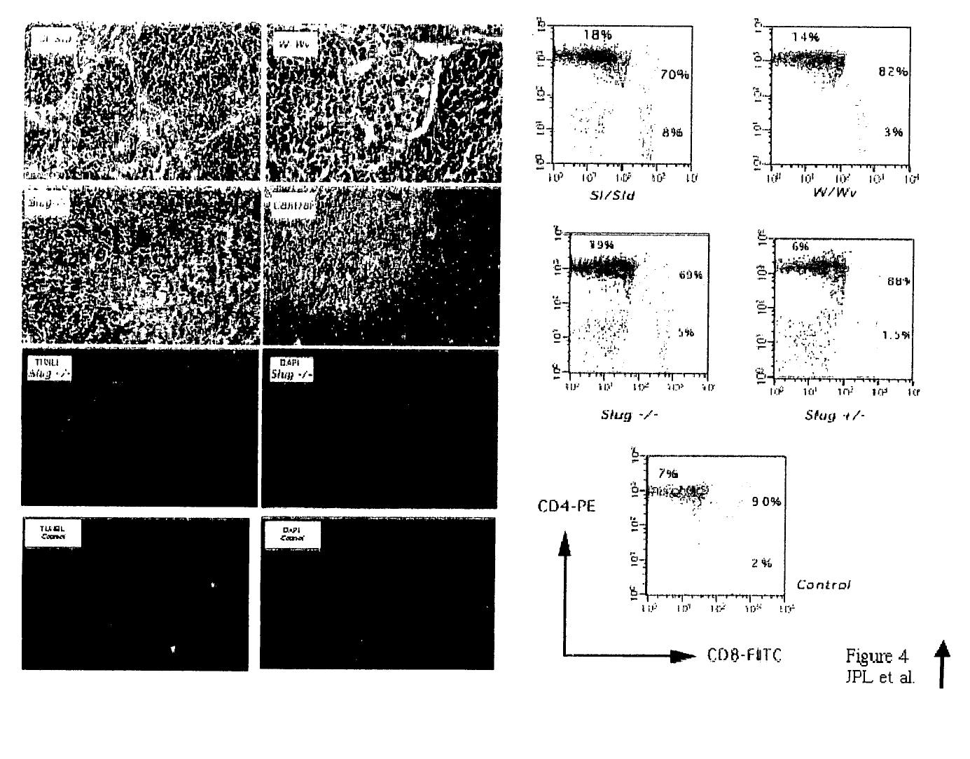

In mice where the functional expression of Slug is missing, the number of T cells in peripheral blood is normal, although an analysis of the composition of the thymus in 4-week old mice showed a reduction in the production of cells and differentiation toward the CD3*CD8* cells that was similar to the mutant W and S1 mice (FIG. 4). This specific blocking of T cell differentiation was observed in +/−mice. The thymus of Slug −/− mice was small and studied in histological sections. Morphological differences were detected between the thymus of 4- and +/+animals from the same litter, the histological appearance being similar to the thymus of mutant S1 and W mice (FIG. 4). In sections of the thymus of Slug-deficient mice, many cells were observed at the cortical level that seemed to belong to apoptopical bodies that are not often seen in thymus sections of wild mice (FIG. 4). According to this interpretation, a significant increase in TUNEL-positive cells was observed in the thymus sections of Slug-deficient mice. The increase in apoptosis in Slug-deficient animals was correlated with the atrophy of the thymus. These results are consistent with the idea that SCF stimulates the growth of CD4*CD8* thymocytes in primitive mice but not CD4*CD8* cells or individual CD4*CD8* cells (J. Immunol 152:4783, 1994; Cell Immunol. 157:118, 1994).

### 3.3. The Development of B Cells, Myeloid Cells and Mastocytes Appears to be Normal in Slug-Mutant Mice

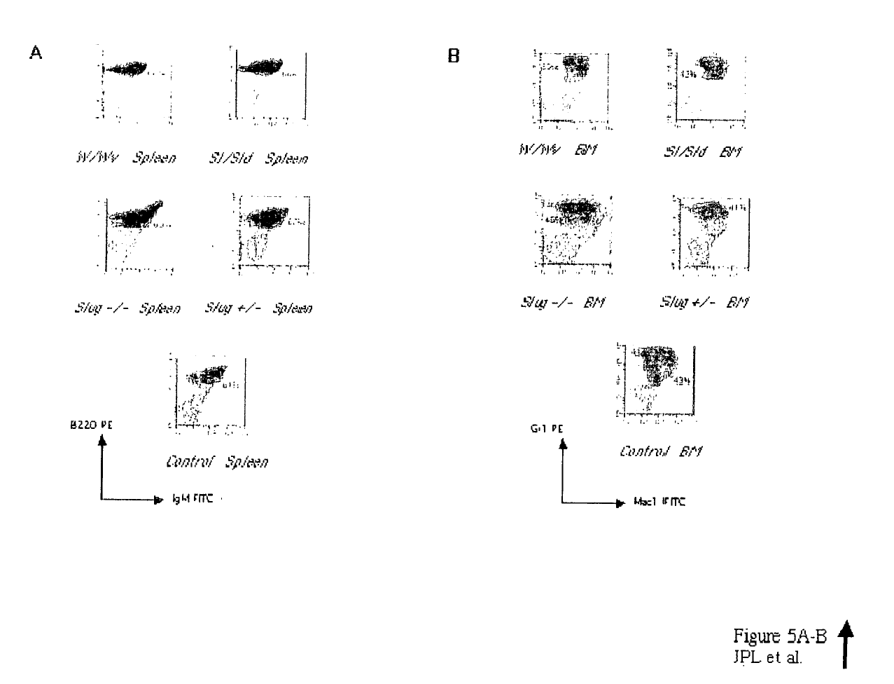

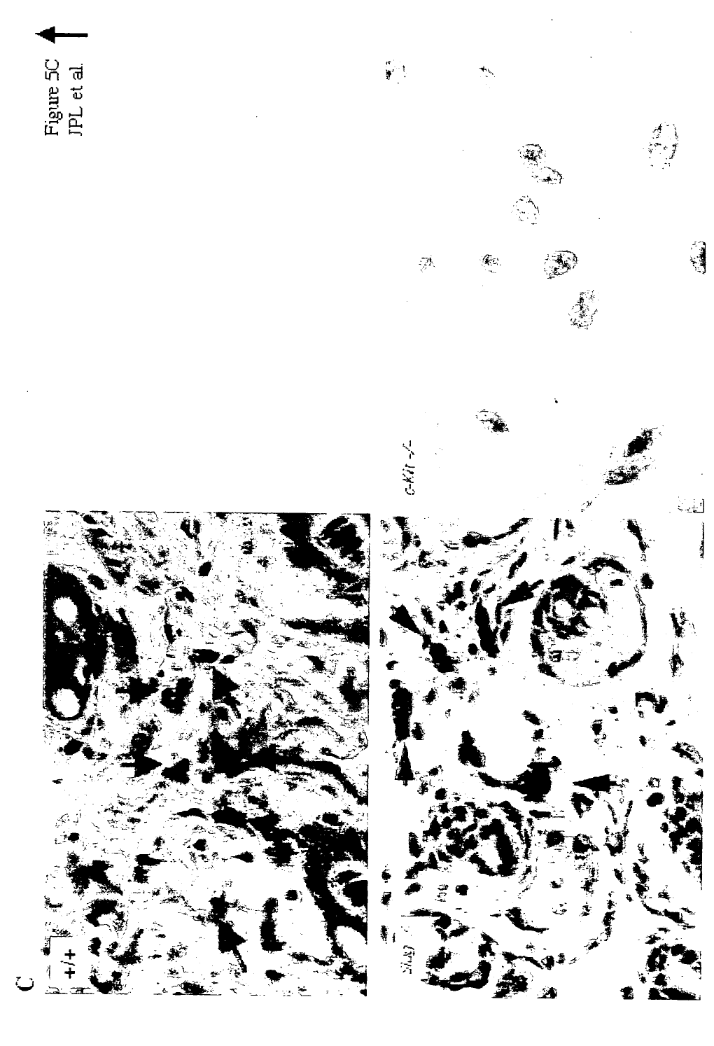

While the interaction between c-kit and SCF is not necessary for the development of B cells and myeloids in vivo, a thorough analysis of expression was conducted using flow cytometry of the differentiation markers on the cell surface in the spleen and bone marrow cells of 5-week old wild mice, mutant Si and W mice and Slug-mutant mice. No reduction in the cells of the myeloid lines and B cells was observed in Slug-mutant mice (FIGS. 5A-B). Therefore, unlike the important function of the Slug gene, like the c-kit/SCF interaction, in the generation of erythroidal lines and T cell, the Slug gene does not appear to be necessary for the normal development of B cells and myeloids in adult mice.

It is well known that the SCF/c-kit signaling route is necessary for the development of mastocytes. The mastocytes of Slug-mutant mice between 4 and 8 weeks old were examined in histological sections of different tissues. No morphological difference was detected between the mastocytes of +/+ and −/− animals from the same litter (FIG. 5C). Moreover, the number of mastocytes in the ear, an organ known to be rich in mastocytes, was the same in+/+ and −/− animals. Consequently, the development and differentiation of mastocytes does not appear to have been affected by the absence of a functional Slug gene.

### The Defect in Slug-Mutant Mice is Intrinsic to the Stem Cell

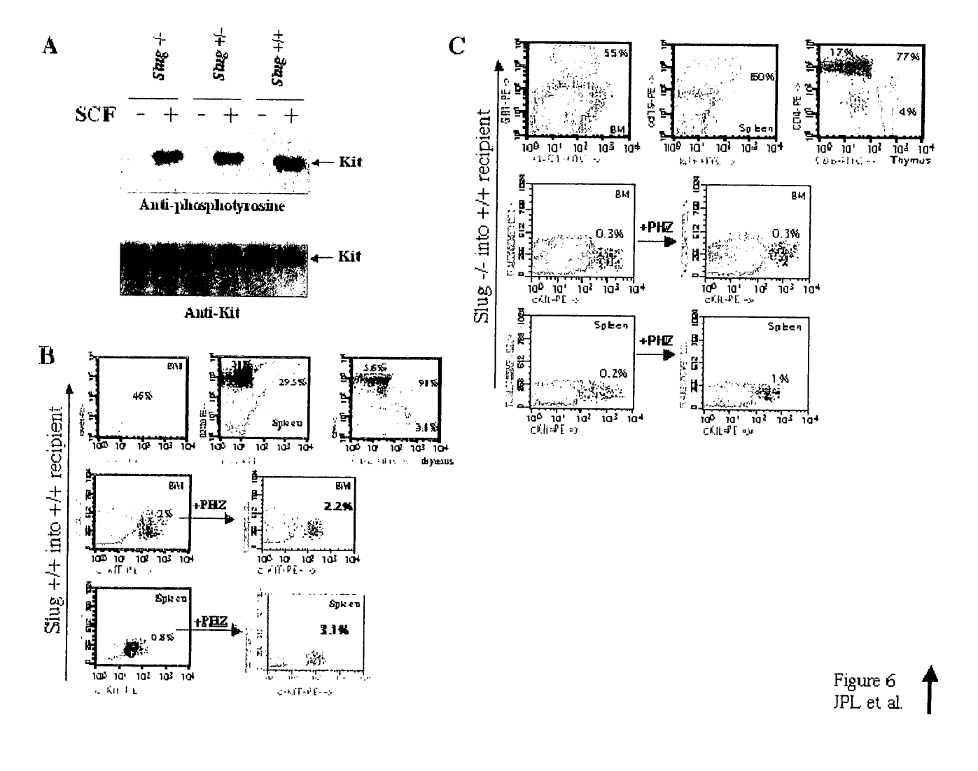

Since the signaling of the receptor depends on the interaction with the linking, it is not surprising to find that mutant forms of the c-kit receptor and its linking produce almost identical developmental defects. However, transplant experiments reveal a significant difference between two mutations: the hematopoyetic stem cells in S1 mice function normally in wild type receptors, while the same cannot be said of mutant W mice. Consequently, since the absence of the Slug gene affects the development of three populations of stem cells: melanoblasts, hematopoyetic stem cells and germinal cells (as in both W and S1 mutations), the Slug-mutant mice were first analyzed to see if they had a normal SCF/c-kit signaling route. To ensure a normal receptor of transmembrane tyrosine kinsase coded by c-kit for SCF (c-kit/SCF-R), the primary mastocytes in the bone marrow of +/+, +/−and −/− mice of the same age were examined. The c-kit/SCF-R pair in −/−, +/−and control mice was the same size and was expressed at comparable levels (FIG. 6A). The c-kit/SCF-R pair was also kinase active and self-phosphorilized tyrosine remains after stimulation with SCF (FIG. 6A).

To define whether the nature of the defect was extrinsic or intrinsic to the stem cell, the capacity of the hematopoyetic stem cells in Slug-mutant mice to reconstitute a hematopoiesis in radiated hosts was analyzed. The grafting of the bone marrow cells from a normal donor cures the hematopoyetic phenotype observed in Slug −/− mice. On the other hand, the wild receptors that had been lethally radiated and reconstituted with hematopoyetic stem cells from Slug −/− mice presented macrocitical anemia and the composition of the hematopoyetic system was similar to that of Slug −/− mice (FIG. 6B). These mice showed normal development of B cells and myeloids, blocking of the differentiation of T cells toward CD4*CD8* cells and when treated with PHZ the c-kit cells could not migrate to the spleen. These results indicate that the defect in Slug-mutant mice is intrinsic to the stem cell.

### Primary c-kit Cells do not Express Slug in Mutant W or S1 Mice

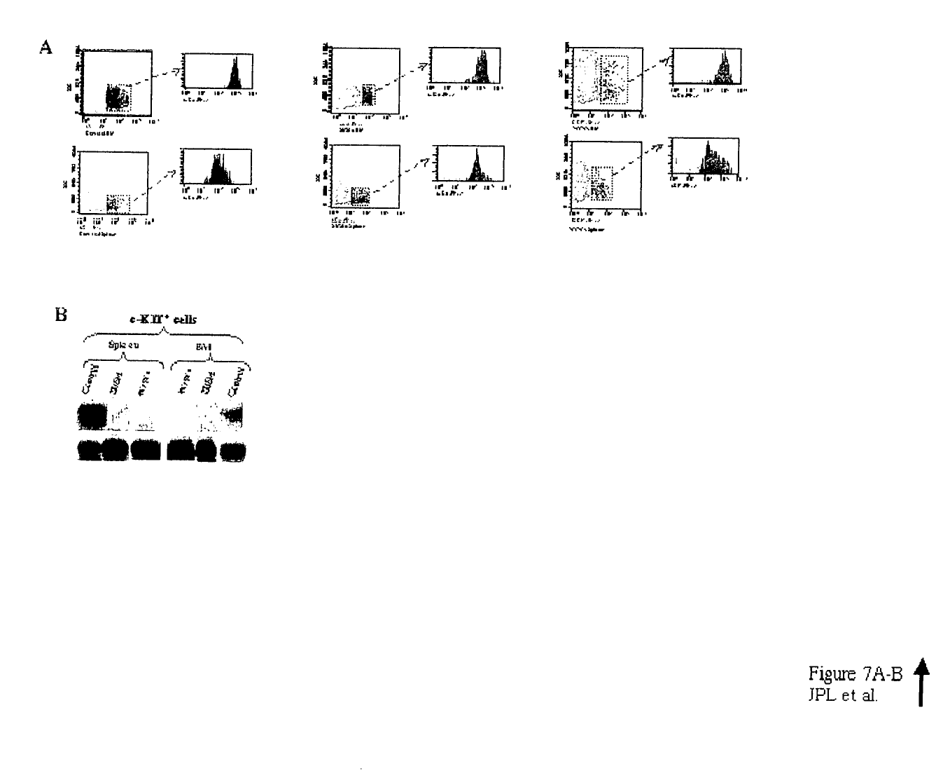

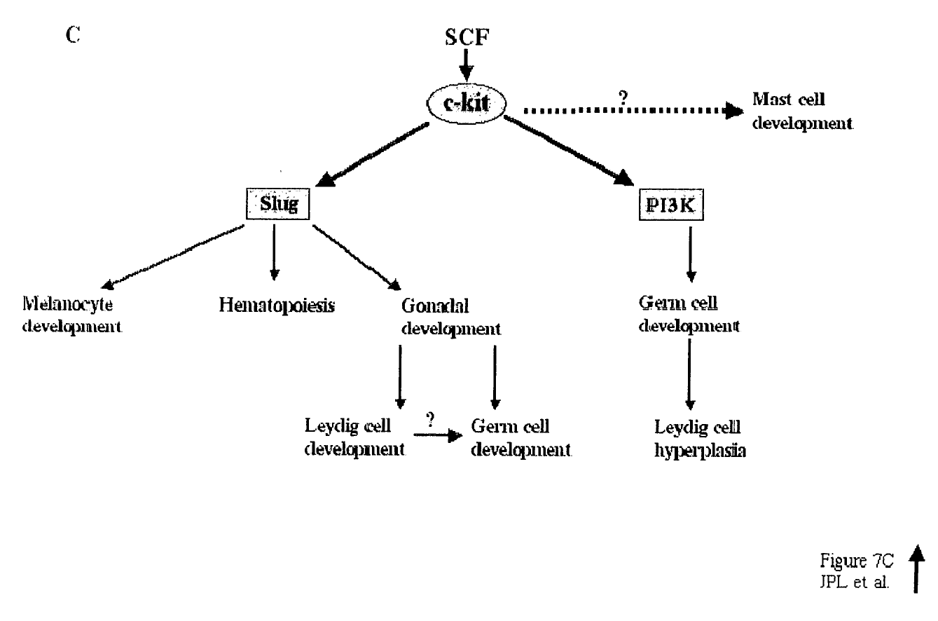

The Slug gene favors the basic functions for promoting the development, survival and proliferation of hematopoyetic stem cells, those derived from the neural crest and germinal cells, a function well illustrated by the reduction of erythroidal precursors and associated macrocitical anemia, gonadal defects and hypopigmentation in Slug-deficient mice. The discovery that the activation of the c-kit receptor specifically induces expression in Slug mice and that Slug-deficient mice have a phenotype similar to that of mutant S1 and W mice led researchers to verify whether the levels of expression of the Slug gene are regulated as a consequence of the activation of SCF/c-kit in control cells as opposed to the primary c-kit cells in S1 and W mice. Hemolytic anemia was induced with phenylhydrazine (PHZ) in control mice and in mutant S1 and W mice. On day 3, the c-kit cells in the bone marrow and spleen were purified, classifying them into control mice and mutant S1 and W mice (FIG. 7A). It was later verified whether the expression of the Slug gene was also present in the c-kit cells of control mice. An examination of the expression of the Slug gen by RT-PCR revealed that the Slug gene was present in the primary c-kit cells derived from the bone marrow and spleens of control mice (FIG. 7B). The expression of β-actin was used to evaluate the integrity and load of each RT-PCR reaction (FIG. 7B, lower section). The expression of the Slug gene was higher on the insides of the migratory cells observed in the spleen than in the c-kit cells that remained in the bone marrow. On the contrary, under the same empirical conditions, the expression of the Slug gene could not be detected in the purified primary c-kit cells in the bone marrow of mutant W and S1 mice. The expression of the Slug gene was only observed in the primary c-kit cells from the spleen (migratory cells) of mutant W mice. These results, along with the discovery that the activation of the c-kit receptor specifically induce Slug gene expression and that Slug-deficient mice have a phenotype similar to that of mutant W and S1 mice indicates that the Slug gen is the molecular objective that provide biological specificity to the SCF/c-kit signaling route.

### III. DISCUSSION

### Defects in the Development of the SCF/c-kit Signaling Route Mediated by Slug

The in vivo SCF/c-kit migratory route and the destinations of development have been well known since 1990, due to the existence of mutant mice in which both the coded genes of the receptor and those of their respective linkings are defective (Nature 335, 88, 1998; Cell 55:185, 1988; Cell 63:225, 1990; Cell 63:203, 1990; Cell 63:167, 1990; Cell 63:75, 1990; Cell 63:213, 1990). However, much less is known about the mechanism that provide biological specificity to the SCF/c-kit signaling route in the formation and migration of c-kit cells. A key aspect is the identification of the c-kit signaling objectives that reinforces the migratory behavior of the c-kit cells. In this regard, the biological events controlled by the c-kit signaling route are similar to those that take place in epithelial-mesenchymal transitions (EMT) during mammal development and are controlled by “zinc finger” type transition factors in the Snail family (Nieto et al, 1994, Science 264:835-849; Cano et al, 2000, Nature Cell Biology 2:76-83). These proteins, which share an evolutionary conservation role in both vertebrates and invertebrates, are involved in the generation and migration of mesodermal cells and the neural crest of numerous vertebrate species. Research has not been conducted to determine whether the biological functions governed by the SCF/c-kit signaling route are mediated by the Snail protein family. The results demonstrate that the activation of the c-kit receptor by its SCF linking specifically induces the expression of Slug, a specific member of the Snail family, indicating a clear relationship between the activation of SCF/c-kit and the expression of the Slug gene.

Due to the fact that the mice with mutations in the c-kit receptor and its linking (SCF) have the same complex phenotype that affects pigmentation, germinal cells ad hematopoiesis, the mice that did not have the Slug gene were analyzed thoroughly to determine in vivo whether the functions of the c-kit/SCF route were mediated by Slug. Mice that experiences mutations with loss of Slug functions were generated. The pattern of expression of the Slug gene suggested that this gene played a role in the development of the nervous system. Consequently, the analysis of mutant mice focused on the nervous system At this time, the analysis of mutant mice is focused on those developmental aspects that depend on the SCF/c-kit route. The results show the presence of defects in dermatological, gonadal and hematopoyetic development in Slug-mutant mice.

Only Slug −/− mice showed alterations in pigmentation, which indicates that the absence of Slug only affects the migration and/or survival of pigmented stem cells derived from the neural crest, i.e., on the forehead and on the extremities. This function of Slug is consistent with the fact that the Slug gene in mice is not expressed in premigratory cells of the neural crest but is expressed in the migratory cells of the neural crest. The alterations observed in the pigmentation of Slug −/− mice are similar to the alterations described in mutant W and S1 mice, which explains why some areas on mutant heterocygotic W and S1 mice and on individuals with piebaldism phenotypes are completely depigmented while others are normal. These data also indicate that the intracellular signaling mediated by c-kit must overcome a critical threshold in order for the Slug to be activated and for the melanoblasts to migrate and survive. The heterocygotic S1 and W mice appear not to reach this threshold. In fact, the melanoblasts that migrate to the forehead and other affected areas may be at the lower end of SCF gradient values (Development 109, 911-923, 1990).

In addition to the melanocyte deficiency, the homocygotic mutant mice for Slug showed testicular defects. These defects involved both sperm and Leydig cells. The sperm defect in homocygotic mutant W and S1 mice, which are sterile, is well known and is controlled by activation with P13-kinase mediated by kit (Blume-Jensen et al, 200, Nature Genetics, 24:157-162; Kissel et al, 2000, EMBO J. 19:1312-1326) However, the interstitial space in homocygotic W and S1 mice and in the testicles of kit^{Y719F}/kit^{Y719F} has increased disproportionately and has filled up with Leydig cells. One possible explanation for these observations is the existence of balancing mechanisms in the Leydig line such as FSH and factor-1 of insulin-type growth which attempt to compensate the deficiencies in the behavior of primitive germinal cells in mutant W and S1 and kit^{Y719F}/kit^{Y719F} mice, stimulating the proliferation or survival of the Leydig cells. On the contrary, in Slug −/− mice, the main problem may lie in the behavior of the Leydig cells derived from neuronal crests and which are c-kit. This deterioration in the development of Leydig cells could, as a side effect, affect the maturation of germinal cells. Therefore, the SCF/c-kit signaling route would have a dual function in the testicle: in the development of the germinal cells controlled by the activation of P13-kinase mediated by kit and in the development of Leydig cells controlled by the Slug gene (FIG. 7C). The analysis of hematopoyetic development in Slug-mutant mice showed a phenotype similar to that of defective W and I mice. The homocygotic mutant S1, W and Slug mice presented macrocitical anemia. These mutations deteriorate the developmental capacity of the progenitor cells of the erythroidal and T cell lines, but show normal development as far as B cells and myeloids are concerned. The defect in the development of hematopoyetic cells in Slug-mutant mice was intrinsic to the cell. The Slug-mutant mice presented equal phenotypes, regardless of whether the hematopoyetic cells were isolated directly from mutant mice or recovered from transplant receptors. Therefore, the phenotype of Slug-mutant mice is not due to insufficiencies in the microenvironment (as in mutant S1 mice) but rather to intrinsic defects in the hematopoyetic cells of the progenitor (as in mutant W mice).

Other lines that express kit, such as mastocytes and most melanoblasts, shown no obvious phenotypes in Slug-mutant mice, which suggests that the cellular context is of paramount importance for interpreting the SCF/c-kit signal. In this type of cells, the Slug function is either not required or can be compensated through the synergetic effect with other members of the Snail family. Another question deals with why the loss of the Slug function from heterocygotic cells produces phenotype abnormalities. This would seem to indicate that a loss of the mutation function in the alelo cannot be offset with the rest of the wild alleles of the same gene, defining Slug as a semidominant gene.

S1, W and Slug mutations affect the development of three cell populations: melanoblasts, hematopoyetic stem cells and germinal cells. Slug is therefore present in migratory c-kit ells and is not present in c-kit cells in the bone marrow of homocygotic S1 and W cells, which demonstrates the role of the Slug gene in c-kit cells acquiring migratory capacity. These results are consistent with the model in which the stem cells containing the c-kit receptor would express the Slug gene, provoking the survival of the cell regardless of the external signal required (SCF) and permitting the cells to migrate outside of their normal environment. If this does not occur within a certain period of time, apoptosis could occur since the cells have been deprived of external signals to retain the expression of the Slug gene. This would prevent migratory cells from entering territories that are unsuitable to their specification status. These data indicate that the signals that regulate cell destination (or cell death) play and important role in maintaining the patterns of cellular specifications and differentiation.

These results identify the Slug gene as a transcription factor that controls the migration and survival of c-kit cells. In this sense, it is known that p53 deficiency rescues androfertility in W mice but does not affect he survival of melanocytes and hematopoyetic cells Therefore, the apoptosis of masculine germinal cells in the absence of c-kit depends on p53 (Dev. Biol. 1999, 215:78-90). The results obtained here show that Slug is the survival factor in melanocyte and hematopoyetic cell lines. The Slug protein normally acts as a repressor (Dev. Biol. 2000, 221:195-205). Slug could therefore regulate the genes whose expression needs to be excluded from c-kit cells in order to migrate.

### Slug is a Candidate Gene of Hereditary Anemia and Piebaldism in Humans

Disorders in the development of melanocytes are characterized by a heterogeneous distribution of pigmentation known as “white spotting”, typified by piebaldism and by the Wardenburg Syndrome. It is now clear that these disorders in the development of cell pigment represent a subgroup of neurochristopathies that involve defects in several cellular lines of the neural crest that include melanocytes. The results obtained herein implicate Slug as the cause of the piebaldism feature. The alteration in the Slug gene may be responsible for the piebaldism phenotype in some cases. Consequently, it may be confirmed that in some patients the piebaldism feature is the result of delections in this gene rather than mutations in the c-kit receptor gene.

Another characteristic of Slug-mutant mice is anemia. Congenital human anemia such as Diamond-Blackfan anemia (DBA), which is characterized by a decrease in the progenitors of the erythroids in the bone marrow, is similar in some ways to the anemia in Slug-mutant mice. However, it was previously observed that humans with pathological mutations of the KIT gene do not present anemia (Spritz, 1992, Blood 79:2497). A direct test of this hypothesis is now viable.

### SCF/c-Kit/Slug in Transformation

The c-kit receptor is implicated in both leukemia and other solid tumors. Mutations that result in the constitutive activation of c-kit have been described in acute myeloid leukemia, in small cell lung cancers, in gynecological cancers, breast carcinoma and in colon tumors derived from Cajal interstitial cells (a type of cell that is dependent on SCF). However, the oncological potential supposedly conferred on alterations in c-kit activity in malignancy is untrue. The results show that Slug confers migratory and survival properties on c-kit cells. Therefore, the constitutive activation of c-kit could confer invasive properties on tumor cells. In this context, Slug may also represent a relevant molecular event in cell transformation. Recent discoveries show that Slug is also expressed in leukemia cells t(17;19) and in rhabdomiosarcoma cells that express the PAX3-FKHR translocation. Slug could therefore be a component of invasion in cancer biology.

### Potential Uses of Slug

The mobilization of hematopoyetic stem cells is important in clinical transplants, gene therapy and in the ex vivo expansion of hematopoyetic stem cells, as well as masculine sterility. However, these and other applications of SCF have been limited by the activating properties of mastocytes (Broudy, 1997, Blood 90:1345-1364). The results provided by this invention identify Slug as the molecule that mediates in the function of the SCF/c-kit signaling route, suggesting that Slug could have the same clinical applications as SCF, with the advantage that Slug would not activate mastocytes.

1. A pharmaceutical composition that comprises the Slug gene, the complementary DNA to the RNA that codes for the product of transcription or expression of the said Slug gene (CDNA), the Slug protein and/or one or more drugs or substances that activate the expression of the Slug gene, along with one or more pharmaceutically acceptable excipients.

2. A pharmaceutical composition according to claim 1 that comprises the Slug gene or the CDNA of the Slug gene on a viral or non-viral vector.

3. Use of the Slug gene, the complementary DNA that codes for the product of transcription or expression of the said Slug gene (CDNA), the Slug protein and/or one or more drugs or substances that activate the expression of the Slug gene to prepare a pharmaceutical composition to mobilize hematopoyetic stem cells for transplants or gene therapy.

4. Use of the Slug gene, the complementary DNA that codes for the product of transcription or expression of the said Slug gene (CDNA), the Slug protein and/or one or more drugs or substances that activate the expression of the Slug gene to prepare a pharmaceutical composition to treat masculine sterility.

5. In accordance with claim 4, use of the Slug gene, CDNA of the said Slug gene, the Slug protein and/or one or more drugs or substances that activate the expression of the Slug gene to prepare a pharmaceutical composition to treat masculine sterility caused by a decrease in Leydig cells.

6. A method for the ex vivo expansion of hematopoyetic stem cells involving the use of the Slug gene, the RNA that codes for the product of transcription or expression of the said Slug gene (CDNA), Slug protein and/or drugs or substances that activate the expression of the Slug gene.

7. A method, in accordance with claim 6, that involves the administration of a culture of hematopoyetic stem cells of an effective quantity of the Slug gene, the Slug gene's DAN, slug protein and/or drugs or substances that activate the expression of the Slug gene or a composition containing such products.


SEQUENCE LISTING (6 sequences)

SEQ ID NO: 1 - DNA, 20 bp, Artificial Sequence
  Direct Oligonucleotide Initiator
gcctccaaaaagccaaacta

SEQ ID NO: 2 - DNA, 20 bp, Artificial Sequence
  Reverse Oligonucleotide Initiator
cacagtgatggggctgtatg

SEQ ID NO: 3 - DNA, 20 bp, Artificial Sequence
  Direct Oligonucleotide Initiator
cagctggccaggctctcggt

SEQ ID NO: 4 - DNA, 18 bp, Artificial Sequence
  Reverse Oligonucleotide Initiator
gcgagggcctccggagca

SEQ ID NO: 5 - DNA, 24 bp, Artificial Sequence
  Internal Probe
gacacacatacagtgattatttcc

SEQ ID NO: 6 - DNA, 29 bp, Artificial Sequence
  Internal Probe
tgcaaccgtgttttgctgaccgctccaac